(19) United States
(12) Patent Application Publication
(10) Pub. No.: US 2005/0107717 A1
(43) Pub. Date: May 19, 2005

(54) Method and device for calculating visceral fat

(75) Inventors: Koji Yamamoto, Kyoto-shi (JP); Tatsuya Kobayashi, Otsu-shi (JP)

Correspondence Address: Barry E. Bretschneider;Morrison & Foerster LLP, Suite 300, 1650 Tysons Boulevard, McLean, VA, 22102, US

(73) Assignee: OMRON HEALTHCARE Co., Ltd,, Kyoto-shi (JP)

(21) Appl. No.: 10/954,640

(22) Filed: Oct. 1, 2004

(30) Foreign Application Priority Data
Oct. 2, 2003 (JP) 2003-344532

(51) Int. Cl. A61B 5/05

(52) U.S. Cl. 600/547

(57) ABSTRACT

A visceral fat area of a subject is calculated based on an equation using as variables a sex of the subject, and an electric impedance ratio between the longitudinal width of the abdomen and the transversal width of the abdomen of the subject. As an example of the equation, let the visceral fat area (cm²) be VFA and the longitudinal-transversal impedance ratio of the abdomen be R1, then VFA=a1+b1×R1 (where a1 and b1 are constants). A visceral fat area of the subject is also calculated based on an equation using abdomen circumference as an additional variable. As an example of the equation, let the abdomen circumference (cm) be W and the longitudinal-transversal impedance ratio of the abdomen be R4, then VFA=W×(a4+b4×R4)+c4 (where a4, b4 and c4 are constants) or VFA=W²×(α4+β4×R4)+γ4 (where α4, β4 and γ4 are constants). The calculated visceral fat area is compared with the reference value, and the comparison results are outputted. In the same manner, visceral fat weight and visceral fat volume can be calculated.

### BACKGROUND OF THE INVENTION

1. Field of the Invention

The present invention relates to a method and device for calculating visceral fat, with the function of calculating a visceral fat area on the abdomen at navel height by the bioimpedance method.

2. Description of the Related Art

In recent years, with an increase in corpulent population, the importance of healthcare has been pointed out and the problem of corpulence has been drawing more and more attention. Above all, clinical findings have made it evident that visceral fat has a close relation with the risk of developing life-style diseases, so that there is a rapidly growing interest in easy and accurate measurement of visceral fat.

In conventional methods and devices for calculating visceral fat with ease, the bioimpedance method is used most. For example, Japanese Patent Application Laid-Open (JP-A) Nos. 11-123182, 2000-175875, 2001-286452 and 2002-315730 disclose measuring impedance by arbitrary arranging electrodes around-the abdomen. However, in the arts described in these documents, impedance is measured at an arbitrary body part, failing to perform accurate measurement of a variety of fat distributions of different subjects, or requiring a large-sized measuring device.

In order to solve these problems, JP-A Nos. 2001-178697, 2001-252256, 2001-252257, 2002-238871 and 2002-369806 disclose measuring impedance at a specific abdomen part so as to calculate a visceral fat area.

### SUMMARY OF THE INVENTION

However, in the arts described in the latter five patent documents, impedance measurements are performed by arranging application electrode pairs in such a manner as to substantially be opposed to each other around the abdomen of the human body, and further arranging measurement electrode pairs in such a manner as to be substantially parallel with or to cross the application electrode pairs. In these measurements, visceral fat is exclusively measured by specifying a positional relation between the electrode pairs to be arranged because no electrophysiological difference is seen between visceral fat and subcutaneous fat. However, the quantity of subcutaneous fat sometimes affects impedance measurement value, thereby further affecting measuring precision.

The present invention, which has been contrived in view of these conventional problems, has an object of providing a method and device for calculating visceral fat easily and accurately by the bioimpedance method.

In order to achieve the above object, the present invention is a visceral fat calculation method comprising the step of calculating a visceral fat area of the subject based on an equation using as variables the sex of the subject, and an electric impedance ratio between the longitudinal width of the abdomen and the transversal width of the abdomen of the subject.

Let the visceral fat area (cm²) be VFA and a longitudinal-transversal impedance ratio of the abdomen be R1, then the equation is expressed as VFA=a1+b1×R1 (where a1 and b1 are constants).

The present invention is a visceral fat calculation method comprising the step of calculating a visceral fat weight of a subject based on an equation using as variables the sex of the subject, and an electric impedance ratio between the longitudinal width of the abdomen and the transversal width of the abdomen of the subject.

Let the visceral fat weight (kg) be VFW and the longitudinal-transversal impedance ratio of the abdomen be R2, then the equation is expressed as VFW=a2+b2×R2 (where a2 and b2 are constants).

The present invention is a visceral fat calculation method comprising the step of calculating a visceral fat volume of a subject based on an equation using as variables the sex of the subject, and an electric impedance ratio between the longitudinal width of the abdomen and the transversal width of the abdomen of the subject.

Let the visceral fat volume (cm³) be VFV and the longitudinal-transversal impedance ratio of the abdomen be R3, then the equation is expressed as VFV=a3+b3×R3 (where a3 and b3 are constants).

The present invention is a visceral fat calculation method comprising the step of calculating a visceral fat area of a subject based on an equation using as variables the sex of the subject, an abdomen circumference at navel height, and an electric impedance ratio between the longitudinal width of the abdomen and the transversal width of the abdomen of the subject.

Let the visceral fat area (cm²) be VFA, the abdomen circumference (cm) be W, and the longitudinal-transversal impedance ratio of the abdomen be R4, then the equation is expressed as VFA=W×(a4+b4×R4)+c4 (where a4, b4 and c4 are constants) or VFA=W²×(α4+β4×R4)+γ4 (where α4, β4 and γ4 are constants).

The present invention is a visceral fat calculation method comprising the step of calculating a visceral fat weight of a subject based on an equation using as variables the sex of the subject, an abdomen circumference at navel height and an electric impedance ratio between the longitudinal width of the abdomen and the transversal width of the abdomen of the subject.

Let the visceral fat weight (kg) be VFW, the abdomen circumference (cm) be W, and the longitudinal-transversal impedance ratio of the abdomen be R5, then the equation is expressed as VFW=W×(a5+b5×R5)+c5 (where a5, b5 and c5 are constants) or VFW=W²×(α5+β5×R5)+γ5 (where α5, β5 and γ5 are constants).

The present invention is a visceral fat calculation method comprising the step of calculating a visceral fat volume of a subject based on an equation using as variables the sex of the subject, an abdomen circumference at navel height, and an electric impedance ratio between the longitudinal width of the abdomen and the transversal width of the abdomen of the subject.

Let the visceral fat volume (cm³) be VFV, the abdomen circumference (cm) be W, and the longitudinal-transversal impedance ratio of the abdomen be R6, then the equation is expressed as VFV=W×(a6+b6×R6)+c6 (where a6, b6 and c6 are constants) or VFV=W²×(α6+β6×R6)+γ6 (where α6, β6 and γ6 are constants).

The present invention is a visceral fat calculation device comprising impedance measurement unit to measure impedances of the longitudinal width of the abdomen and the transversal width of the abdomen of a subject; a longitudinal-transversal impedance ratio of the abdomen calculation unit to calculate the longitudinal-transversal impedance ratio of the abdomen from measured impedances of the longitudinal and transversal widths of the abdomen; subject's information input unit to enter the sex of the subject; subject's information storage unit to store the sex of the subject; visceral fat information calculation unit to calculate visceral fat information by using the longitudinal-transversal impedance ratio of the abdomen and the subject's information; and visceral fat information output unit to output visceral fat information obtained by the visceral fat information calculation unit.

The visceral fat information is one of a visceral fat area, a visceral fat weight or a visceral fat volume.

The present invention is a visceral fat calculation device comprising impedance measurement unit to measure impedances of the longitudinal width of the abdomen and the transversal width of the abdomen of a subject; a longitudinal-transversal impedance ratio of the abdomen calculation unit to calculate the longitudinal-transversal impedance ratio of the abdomen from measured impedances of the longitudinal and transversal widths of the abdomen; subject's information input unit to enter the sex of the subject and an abdomen circumference at navel height; subject's information storage unit to store the sex of the subject and the abdomen circumference at navel height; visceral fat information calculation unit to calculate visceral fat information by using the longitudinal-transversal impedance ratio of the abdomen and the subject's information; and visceral fat information output unit to output visceral fat information obtained by the visceral fat information calculation unit.

The visceral fat information is one of a visceral fat area, a visceral fat weight and a visceral fat volume.

The impedance measurement unit is provided with four or eight application electrodes in pairs and four or eight measurement electrodes in pairs, and the application electrode pairs are disposed to substantially face each other, and the measurement electrodes pairs are also disposed to substantially face each other in such a manner as to sandwich the abdomen therebetween.

The application electrode pairs and the measurement electrode pairs of the impedance measurement unit are disposed on a belt in such a manner as to be slidable in accordance with the abdomen circumference of the subject.

The application electrode pairs and the measurement electrode pairs of the impedance measurement unit are applied with adhesive gel so as to be able to change their positions in accordance with the abdomen circumference of the subject.

The application electrode pairs and the measurement electrode pairs of the impedance measurement unit are provided with means for adhering by a negative pressure so as to be able to change their positions in accordance with the abdomen circumference of the subject.

The visceral fat information output unit outputs comparison results with respect to a predetermined reference value of the visceral fat area.

The predetermined reference value of the visceral fat area is 100 cm².

By the method for calculating visceral fat according to the present invention, visceral fat information can be calculated easily and accurately. The present invention also provides a miniature and inexpensive device for calculating visceral fat.

### BRIEF DESCRIPTION OF THE DRAWINGS

FIG. 1 is a block diagram showing the schematic structure of a device for calculating visceral fat according to an embodiment of the present invention.

FIG. 2 is a flowchart depicting the procedure of a method for calculating visceral fat according to the embodiment of the present invention.

FIG. 3 is a schematic view showing the device for calculating visceral fat according to the embodiment of the present invention which is operated with four electrodes.

FIG. 4 is a schematic view showing the device for calculating visceral fat according to the embodiment of the present invention which is operated with eight electrodes.

FIG. 5 is an example of a printout of calculation results of a visceral fat area.

FIG. 6 is a graph showing the correlation between a visceral fat area in a male person obtained by X-ray CT and a visceral fat area calculated by the method for calculating visceral fat according to the embodiment of the present invention.

FIG. 7 is a graph showing the correlation between a visceral fat area in a female person obtained by X-ray CT and a visceral fat area calculated by the method for calculating visceral fat according to the embodiment of the present invention.

FIG. 8 is an outer perspective view of an example of a belt-type device for calculating visceral fat.

FIG. 9 is an outer perspective view of another example of a belt-type device for calculating visceral fat.

### DESCRIPTION OF THE PREFERRED INVENTION

Embodiments and examples of the present invention will be described as follows.

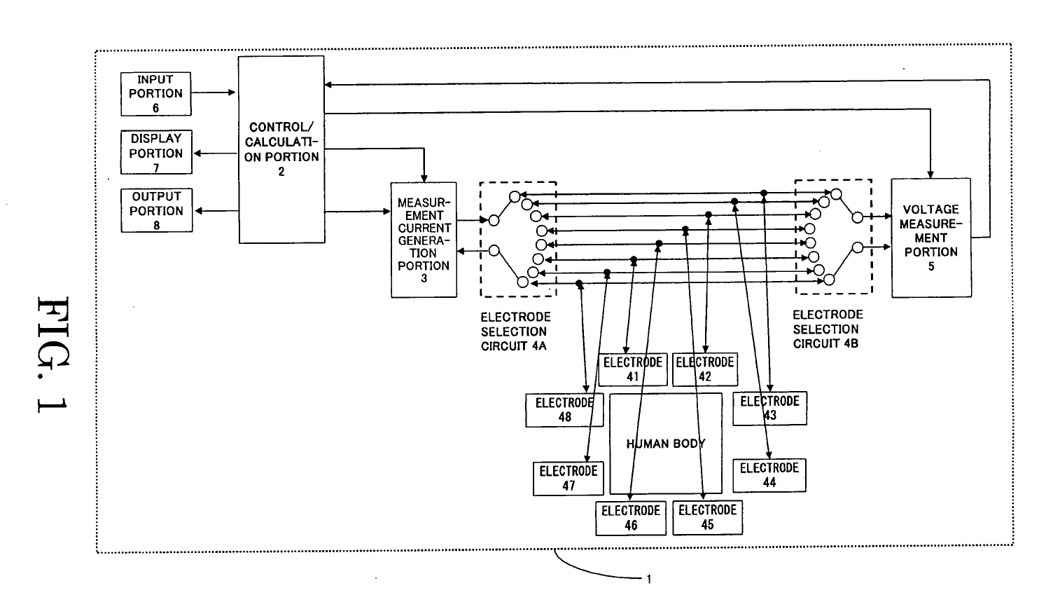

FIG. 1 is a block diagram showing the schematic structure of a visceral fat calculation device 1 according to an embodiment of the present invention.

The visceral fat calculation device 1 includes a control/calculation portion 2, a measurement current generation portion 3, electrode selection circuits 4A and 4B, a group of electrodes 41 to 48, a voltage measurement portion 5, an input portion 6, a display portion 7 and an output portion 8.

The control/calculation portion 2 transmits control signals to the measurement current generation portion 3, the input portion 6, the display portion 7 and the output portion 8 so as to control the measuring operation of the visceral fat calculation device 1. The control/calculation portion 2 receives subject's information from the input portion 6, and stores it in an unillustrated RAM (subject's information storage unit). The control/calculation portion 2 reads out information stored in the RAM, so as to display it on the display portion 7 or to output it to the output portion 8 after calculating visceral fat information based on a predetermined equation. The control/calculation portion 2 has an unillustrated ROM for storing visceral fat calculation equations or programs to define a procedure of impedance measurement operation, which will be described later. The input portion (subject's information input unit) 6 can be any of a keyboard type, a ten key type, a touch screen type, a function key type, etc.

The display of information or results is done on the display portion 7. The output portion (visceral fat information output unit) 8 provides printout of results.

In the visceral fat calculation device 1, when the subject's sex is entered in advance as the subject's information and impedances of the longitudinal and transversal widths of the abdomen of the subject have been measured, the sex and a longitudinal-transversal impedance ratio of the abdomen are used to calculate visceral fat information at navel height, based on a predetermined equation. In addition, a visceral fat area which is an example of the obtained visceral fat information at navel height is compared with an area of 100 cm² advocated by Japan Society for the Study of Obesity, and its results are outputted.

Alternatively, in the visceral fat calculation device 1, when the subject's sex and abdomen circumference at navel height are inputted in advance as the subject's information, and the impedances of the longitudinal and transversal widths of the abdomen have been measured, the subject's information and the longitudinal-transversal impedance ratio of the abdomen are used to calculate visceral fat information at navel height, based on a predetermined equation. In addition, a visceral fat area which is an example of the obtained visceral fat information at navel height is compared with an area of 100 cm² advocated by Japan Society for the Study of Obesity, and its results are outputted.

The impedance measurement unit of an embodiment of the present invention has the group of electrodes 41 to 48, the electrode selection circuits 4A and 4B, the measurement current generation portion 3 and the control/calculation portion 2. The longitudinal-transversal impedance ratio of the abdomen calculation unit and the visceral fat information calculation unit of the present invention are formed of the control/calculation portion 2.

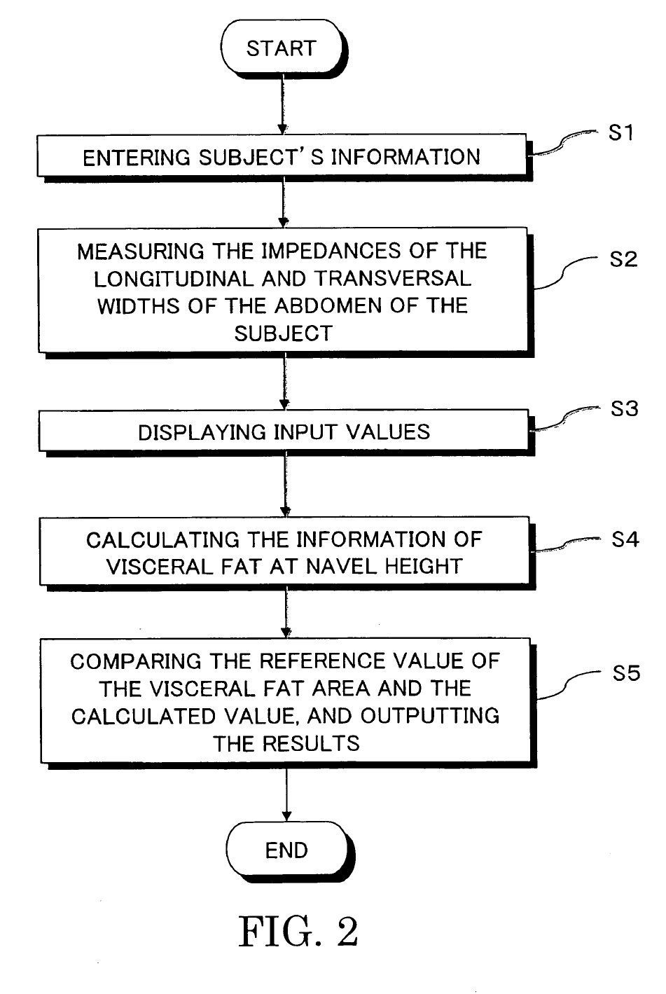

The following is a description of the operation of an embodiment of the present invention. As shown in FIG. 2, first of all, at Step 1 (abbreviated as S1 in the drawing), the sex of the subject which is an example of the subject's information is entered to the visceral fat calculation device 1. Alternatively, it is possible to enter as the subject's information the sex and abdomen circumference. In this case, the abdomen circumference is measured with a measure. The sex, or the sex and the abdomen circumference as the subject's information are stored after being entered.

At Step 2, the impedances of the longitudinal and transversal widths of the abdomen are measured. In the impedance measurement of the longitudinal width of the abdomen, an alternating current is applied between the navel vicinity and the back at navel height, thereby measuring impedance at this part of the body. In the impedance measurement of the transversal width of the abdomen, an alternating current is applied between right and left sides of the abdomen at navel height, thereby measuring impedance at this part of the body.

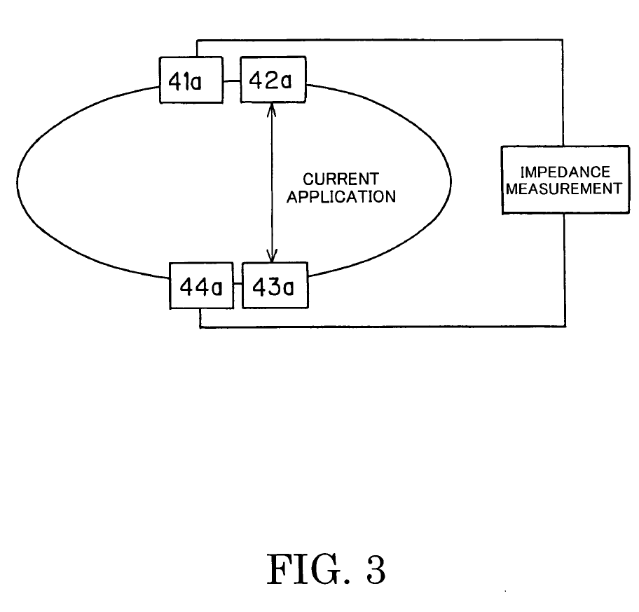

These measurements require four or eight application and measurement electrodes which are respectively arranged in pairs. For example, in an embodiment using four electrodes as shown in FIG. 3, a pair of application electrodes 42a and 43a are disposed to substantially face each other, and a pair of measurement electrodes 41a and 44a are also disposed to substantially face each other with the longitudinal width of the abdomen therebetween, thereby performing a first measurement. To be more specific, an alternating current is applied between the application electrode pair 42a and 43a, and a voltage is detected by the measurement electrode pair 41a and 44a, thereby measuring impedance. After the impedance measurement value of the longitudinal width of the abdomen thus measured is entered, the application electrode pair 42a and 43a are disposed to substantially face each other, and the measurement electrode pair 41a and 44a are also disposed to substantially face each other with the transversal width of the abdomen therebetween, thereby performing a second measurement. The impedance measurement value of the transversal width of the abdomen thus measured is entered in the same manner.

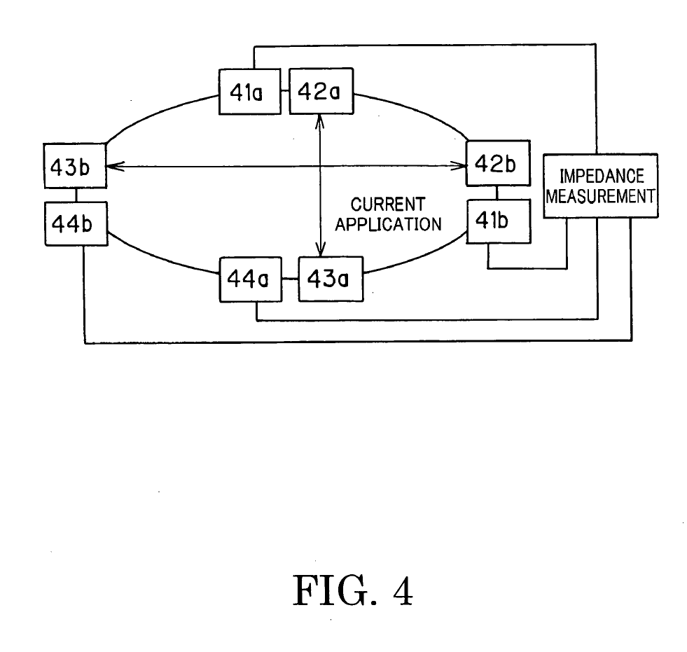

In an embodiment with eight electrodes as shown in FIG. 4, the application electrode pair 42a and 43a are disposed to substantially face each other, and the measurement electrode pair 41a and 44a are also disposed to substantially face each other with the longitudinal width of the abdomen therebetween. Another pair of application electrodes 42b and 43b are disposed to substantially face each other, and another pair of measurement electrodes 41b and 44b are also disposed to substantially face each other with the transversal width of the abdomen therebetween. Measurements are performed either in a time division fashion or at the same time, and the measured values are entered and stored automatically in the device.

At Step 3, the information entered so far is displayed on the display portion 7 of the visceral fat calculation device 1, and recognized by the measurer. In this case, when necessary (for example, in a case that the measured value shows an abnormal value exceeding predetermined upper or lower limit values), the process can return to the previous step to measure impedance again.

At Step 4, with the impedance measurement value and the sex, or the sex and abdomen circumference as the subject's information entered and stored in the visceral fat calculation device 1, visceral fat information is calculated based on a predetermined equation.

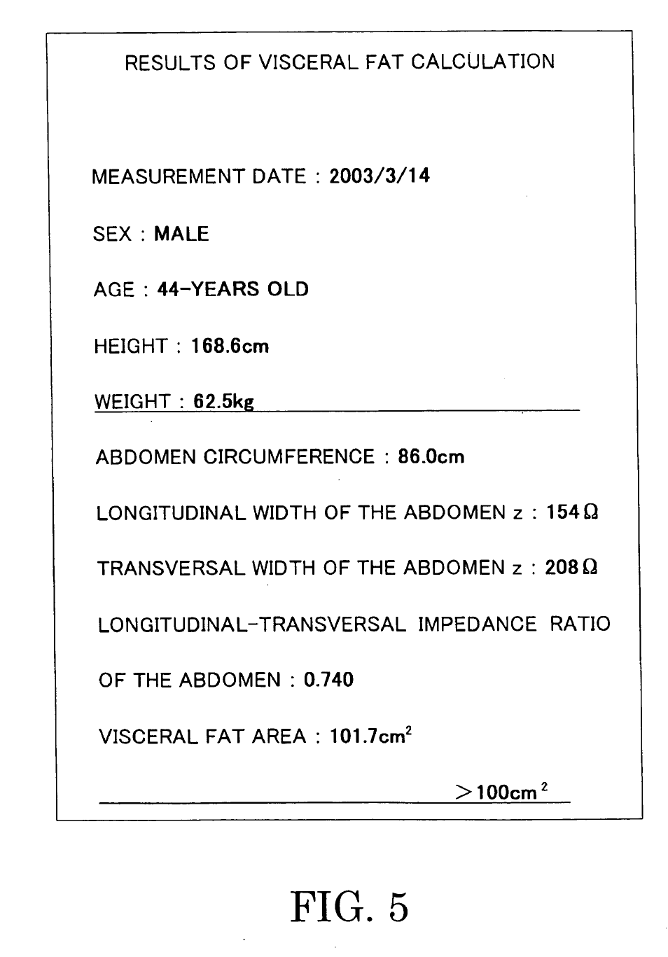

At Step 5, it is determined whether or not the visceral fat area, which is an example of the calculated visceral fat information, is larger than an area of 100 cm² advocated by Japan Society for the Study of Obesity, and the results are outputted by either the unillustrated display portion 7 or the output portion 8 such as a printer. FIG. 5 shows an example of the output form containing measurement date; the sex, age, height, weight and abdomen circumference which have been entered as the subject' information; the measured impedance values of the longitudinal and transversal widths of the abdomen, the longitudinal-transversal impedance ratio of the abdomen calculated from these measured values; and the size relation between the visceral fat area calculated from the subject's information and the measured values, and its reference value. The information that the visceral fat calculation device 1 can provide is not limited to these. It is possible to calculate a body fat rate (quantity), a subcutaneous fat area (quantity), etc. from the impedance values of the longitudinal and transversal widths of the abdomen, so that this information can be displayed or outputted. It is also possible to calculate a V/S ratio, which will be described later, and to display or output it.

The following is a description of the algorism of visceral fat information calculation of the present invention.

Conventionally, as simple clinical indexes of visceral fat, abdomen circumference and V/S ratio have been frequently used. However, in its obesity diagnosis standard, Japan Society for the Study of Obesity established a relation between a flow leading to a diagnosis of obesity and body mass index (BMI), abdomen circumference, visceral fat area, etc. To be more specific, if the BMI is 25 or larger, the abdomen circumference is 85 cm or larger for men and 90 cm or larger for women, and the visceral fat area is 100 cm² or larger, then the person is diagnosed as obese.

In the study of the visceral fat information calculation method according to the present invention, the correlation between the visceral fat area and the abdomen circumference at navel height which is used as an obesity diagnosis standard has been checked to find that the visceral fat area at navel height obtained by X-ray CT which is an antithesis, and the abdomen circumference have a correlation coefficient of about 0.8 for men and about 0.7 for women. When a ratio between a visceral fat area and a subcutaneous fat area which are obtained from measurement by X-ray CT, that is, a V/S ratio is used in addition to the abdomen circumference at navel height, the obtained correlation coefficient has been about 0.95 for men and about 0.88 for women. However, the V/S ratio is obtainable only by measurement by X-ray CT or MRI, which is far from easy and safe measurement.

Therefore, the inventors of the present invention have performed a multiple regression analysis by using abdomen circumference and longitudinal-transversal impedance ratio of the abdomen, and by also using the longitudinal-transversal widths ratio of the abdomen to be obtained from measurement by either X-ray CT or MRI as a variable in place of the V/S ratio. Then, the inventors have made comparison to find that there is a high correlation with the visceral fat area, and that there is also a high correlation between the V/S ratio and the longitudinal-transversal widths ratio of the abdomen. The inventors have also found that there is a high correlation between the longitudinal and transversal widths of the abdomen and the impedance measurement values of the longitudinal and transversal widths of the abdomen. These high correlations are because there is no electrical difference between visceral fat and subcutaneous fat, so that the longitudinal or transversal width of the abdomen can be substantially replaced by the impedance measurement values which reflect the sum of the thickness of visceral fat and the thickness of subcutaneous fat.

The multiple regression analysis has been performed again by using the abdomen circumference at navel height and the longitudinal-transversal impedance ratio of the abdomen as variables.

Let the visceral fat area (cm²) be VFA, the abdomen circumference (cm) be W, and the longitudinal-transversal impedance ratio of the abdomen be R,
then, VFA=W×(a+b×R)+c (where a, b and c are constants), or [Equation 1]
VFA=W²×(a+β×R)+γ (where α, β and γ are constants). [Equation 2]

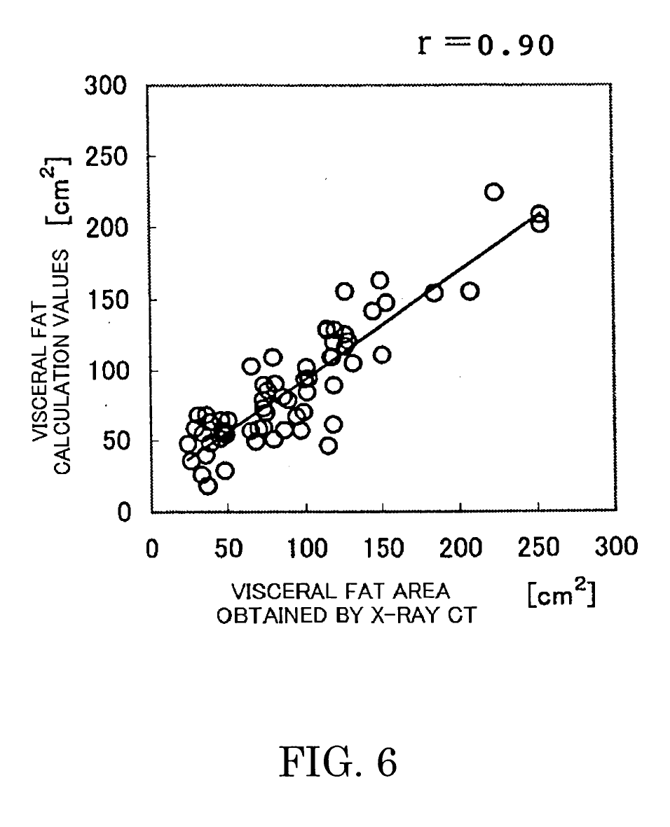

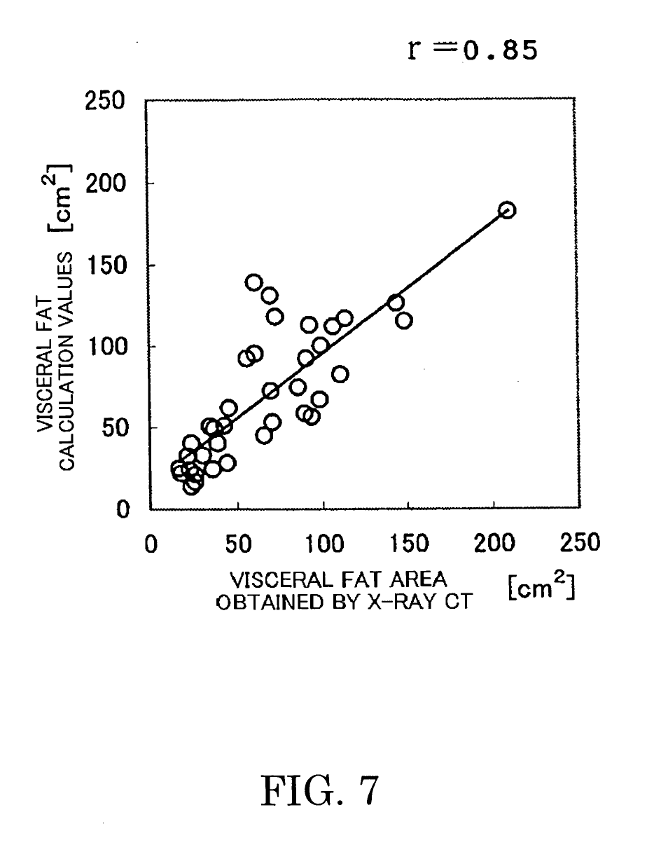

The obtained results of the correlation coefficients are 0.90 for a male as shown in FIG. 6, and 0.85 for a female as shown in FIG. 7.

The aforementioned algorism has been explained by using abdomen circumference; however, in an algorism not using abdomen circumference, let the visceral fat area (cm²) be VFA and the longitudinal-transversal impedance ratio of the abdomen be R,
then, VFA=a+b×R (where a and b are constants). [Equation 3]

As the visceral fat information, the visceral fat area can be replaced by visceral fat weight or visceral fat volume. The visceral fat weight and the visceral fat volume are respectively found as a difference between the total fat weight and the subcutaneous fat weight, and as a difference between the total volume and the subcutaneous fat volume, according to the method of Abe, Fukunaga et al. It is also possible to use as the visceral fat information, visceral fat rate (=visceral fat weight/body weight).

In a case of using as the visceral fat information, visceral fat weight, let the visceral fat weight (kg) be VFW,
then, VFW=W×(a′+b′×R)+c′ (where a′, b′ and c′ are constants), and [Equation 4]
VFW=W²×(α′+β′×R)+γ′ (where α′, β′ and γ are constants). [Equation 5]

In a case of not using the abdomen circumference,
VFW=a′+b′×R (where a′ and b′ are constants). [Equation 6]

In a case of using as the visceral fat information, visceral fat volume, let the visceral fat volume (cm³) be VFV,
then, VFV=W×(a″+b″×R)+c″ (where a″, b″ and c″ are constants) or [Equation 7]
VFV=W²×(α″+β″×R)+γ″ (where α″, β″ and γ″ are constants). [Equation 8]

In a case of using no abdomen circumference,
VFV=a″+b″×R (where a″ and b″ are constants). [Equation 9]

The following is a description of an example of the visceral fat calculation device according to an embodiment of the present invention.

The visceral fat calculation device 1 includes application electrode pairs and measurement electrode pairs arranged on a belt. The method for calculating the visceral fat information is described above, so its description will not be repeated here.

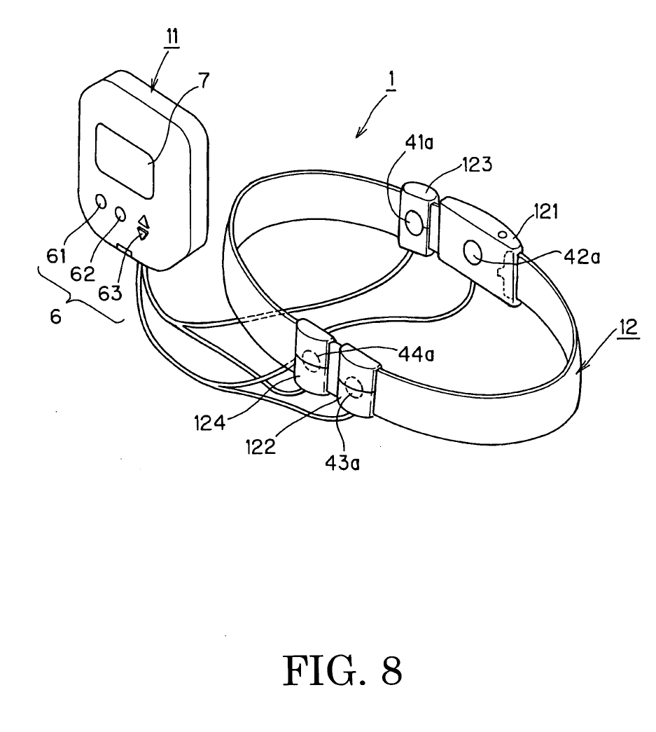

FIG. 8 shows the outer appearance of the visceral fat calculation device 1.

The visceral fat calculation device 1 is formed of a main body 11 and a belt portion 12. The user is supposed to hold the main body 11 and wear the belt portion 12 around his/her abdomen, preferably at navel height.

The main body 11 is provided with an input portion 6 having a power switch 61, a measuring switch 62 and a setting switch 63, and a display portion 7.

Each electrode in the belt portion 12 is connected with the main body 11.

The belt portion 12 has four electrode supporting units 121, 122, 123 and 124 arranged. As an example of arrangement, the electrode supporting unit 121 on the front side of the subject's body and the electrode supporting unit 122 on the back side of the subject's body are provided with application electrodes 42a and 43a, respectively. The electrode supporting units 123 and 124 on the left side of these electrode supporting units 121 and 122 are provided with measurement electrodes 41a and 44a, respectively. The application electrodes 42a and 43a and the measurement electrodes 41a and 44a are arranged in such a manner as to be exposed on the inner surface side of the belt portion 12, thereby coming into contact with the abdomen surface of the subject.

In the present embodiment, the electrode supporting units 122, 123 and 124 can be slid along the belt portion 12, but the electrode supporting unit 121 is integral with the buckle and fixed on the belt portion 12. It goes without saying that this electrode supporting unit 121 can be designed to be slidable along the belt portion 12.

In the belt portion 12, the electrode supporting unit 121 on the front side makes a buckle, so that the length of the belt portion 12 can be adjusted so as to allow a tongue portion (not illustrated) formed at the end to be connected with the electrode supporting unit 121.

The user is supposed to enter predetermined information and to direct operations on the input portion 6. Based on the information entered through the input portion 6, the control/calculation portion 2 performs necessary processes and controls each part. The information including measurement results obtained from the control/calculation portion 2 is displayed on the display portion 7.

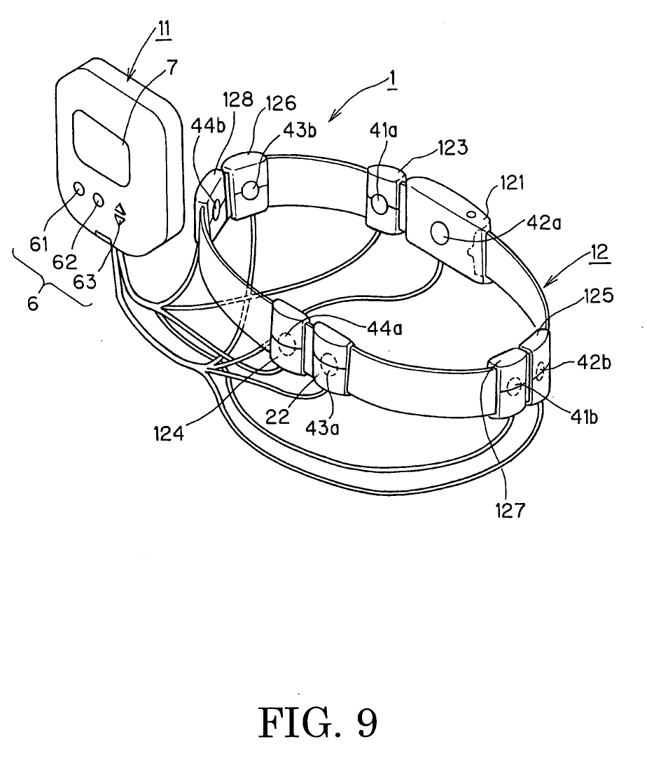

The visceral fat calculation device 1, which includes eight electrode units 121, 122, 123, 124, 125, 126, 127 and 128 arranged on the belt portion 12, is shown in FIG. 9. The electrode supporting units 125 and 126 on the right side of the subject's body are provided with application electrodes 42b and 43b, respectively. The electrode supporting units 127 and 128 on the left side of the subject's body are provided with measurement electrodes 41b and 44b, respectively. The other structure is identical to that of the device with four electrodes shown in FIG. 8.

In the visceral fat calculation device 1 described above, the application electrode pairs and the measurement electrode pairs are disposed on the belt. Instead of this structure, these application and measurement electrode pairs can be fit on the skin of the subject using an adhesive gel so as to be slid freely. It is also possible to dispose the application and measurement electrode pairs on means for adhering to the body surface of the subject by suction with a negative pressure, thereby allowing these electrode pairs to be slid freely. Although the aforementioned visceral fat calculation device 1 has one or two pairs of application electrodes and one or two pairs of measurement electrodes, the numbers of these electrode pairs are not limited to these.

Thus, the visceral fat calculation device 1 can, calculate and provide visceral fat information such as visceral fat area easily and accurately.

1. A visceral fat calculation method comprising:
the step of calculating a visceral fat area of a subject based on an equation using as variables a sex of the subject, and an electric impedance ratio between a longitudinal width of the abdomen and a transversal width of the abdomen of the subject.

2. A visceral fat calculation method according to claim 1, wherein let the visceral fat area (cm²) be VFA and a longitudinal-transversal impedance ratio of the abdomen be R1, then the equation is expressed as VFA=a1+b1×R1 (where a1 and b1 are constants).

3. A visceral fat calculation method comprising:
the step of calculating a visceral fat weight of a subject based on an equation using as variables a sex of the subject, and an electric impedance ratio between a longitudinal width of the abdomen and a transversal width of the abdomen of the subject.

4. A visceral fat calculation method according to claim 3, wherein
let the visceral fat weight (kg) be VFW and a longitudinal-transversal impedance ratio of the abdomen be R2, then the equation is expressed as VFW=a2+b2×R2 (where a2 and b2 are constants).

5. A visceral fat calculation method comprising:
the step of calculating a visceral fat volume of a subject based on an equation using as variables a sex of the subject, and an electric impedance ratio between a longitudinal width of the abdomen and a transversal width of the abdomen of the subject.

6. A visceral fat calculation method according to claim 5, wherein
let the visceral fat volume (cm³) be VFV and a longitudinal-transversal impedance ratio of the abdomen be R3, then the equation is expressed as VFV=a3+b3×R3 (where a3 and b3 are constants).

7. A visceral fat calculation method comprising:
the step of calculating a visceral fat area of a subject based on an equation using as variables a sex of the subject, an abdomen circumference at navel height, and an electric impedance ratio between a longitudinal width of the abdomen and a transversal width of the abdomen of the subject.

8. A visceral fat calculation method according to claim 7, wherein
let the visceral fat area (cm²) be VFA, the abdomen circumference (cm) be W, and a longitudinal-transversal impedance ratio of the abdomen be R4, then the equation is expressed as
VFA=W×(a4+b4×R4)+c4 (where a4, b4 and c4 are constants) or
VFA=W²×(α4+β4×R4)+γ4 (where α4, β4 and γ4 are constants).

9. A visceral fat calculation method comprising:
the step of calculating a visceral fat weight of a subject based on an equation using as variables a sex of the subject, an abdomen circumference at navel height and an electric impedance ratio between a longitudinal width of the abdomen and a transversal width of the abdomen of the subject.

10. A visceral fat calculation method according to claim 9, wherein
let the visceral fat weight (kg) be VFW, the abdomen circumference (cm) be W, and a longitudinal-transversal impedance ratio of the abdomen be R5, then the equation is expressed as
VFW=W×(a5+b5×R5)+c5 (where a5, b5 and c5 are constants) or
VFW=W²×(α5+β5×R5)+γ5 (where α5, β5 and γ5 are constants).

11. A visceral fat calculation method comprising:
the step of calculating a visceral fat volume of a subject based on an equation using as variables a sex of the subject, an abdomen circumference at navel height, and an electric impedance ratio between a longitudinal width of the abdomen and a transversal width of the abdomen of the subject.

12. A visceral fat calculation method according to claim 11, wherein
let the visceral fat volume (cm³) be VFV, the abdomen circumference (cm) be W, and a longitudinal-transversal impedance ratio of the abdomen be R6, then the equation is expressed as
VFV=W×(a6+b6×R6)+c6 (where a6, b6 and c6 are constants) or
VFV=W²×(α6+β6×R6)+γ6 (where α6, β6 and γ6 are constants).

13. A visceral fat calculation device comprising:
impedance measurement unit to measure impedances of a longitudinal width of an abdomen and a transversal width of the abdomen of a subject;
a longitudinal-transversal impedance ratio of the abdomen calculation unit to calculate a longitudinal-transversal impedance ratio of the abdomen from measured impedances of the longitudinal and transversal widths of the abdomen;
subject's information input unit to enter a sex of the subject;
subject's information storage unit to store the sex of the subject;
visceral fat information calculation unit to calculate visceral fat information by using the longitudinal-transversal impedance ratio of the abdomen and the subject's information; and
visceral fat information output unit to output visceral fat information obtained by the visceral fat information calculation unit.

14. A visceral fat calculation device according to claim 13, wherein
the visceral fat information is one of a visceral fat area, a visceral fat weight and a visceral fat volume.

15. A visceral fat calculation device comprising:
impedance measurement unit to measure impedances of a longitudinal width of an abdomen and a transversal width of the abdomen of a subject;
a longitudinal-transversal impedance ratio of the abdomen calculation unit to calculate a longitudinal-transversal impedance ratio of the abdomen from measured impedances of the longitudinal and transversal widths of the abdomen;
subject's information input unit to enter a sex of the subject and an abdomen circumference at navel height;
subject's information storage unit to store the sex of the subject and the abdomen circumference at navel height;
visceral fat information calculation unit to calculate visceral fat information by using the longitudinal-transversal impedance ratio of the abdomen and the subject's information; and
visceral fat information output unit to output visceral fat information obtained by the visceral fat information calculation unit.

16. A visceral fat calculation device according to claim 15, wherein
the visceral fat information is one of a visceral fat area, a visceral fat weight and a visceral fat volume.

17. A visceral fat calculation device according to any one according to any one of claims 13 to 16, wherein
the impedance measurement unit is provided with four or eight application electrodes in pairs and four or eight measurement electrodes in pairs, and the application electrode pairs are disposed to substantially face each other, and the measurement electrodes pairs are also disposed to substantially face each other in such a manner as to sandwich the abdomen therebetween.

18. A visceral fat calculation device according to any one according to any one of claims 13 to 16, wherein
the application electrode pairs and the measurement electrode pairs of the impedance measurement unit are disposed on a belt in such a manner as to be slidable in accordance with the abdomen circumference of the subject.

19. A visceral fat calculation device according to any one according to any one of claims 13 to 16, wherein
the application electrode pairs and the measurement electrode pairs of the impedance measurement unit are applied with adhesive gel so as to be able to change their positions in accordance with the abdomen circumference of the subject.

20. A visceral fat calculation device according to any one according to any one of claims 13 to 16, wherein the application electrode pairs and the measurement electrode pairs of the impedance measurement unit are provided with means for adhering by a negative pressure so as to be able to change their positions in accordance with the abdomen circumference of the subject.

21. A visceral fat calculation device according to any one according to any one of claims 13 to 16, wherein the visceral fat information output unit outputs comparison results with respect to a predetermined reference value of the visceral fat area.

22. A visceral fat calculation device according to claim 21, wherein
the predetermined reference value of the visceral fat area is 100 cm².
